(19) United States
(12) Patent Application Publication
(10) Pub. No.: US 2011/0023893 A1
(43) Pub. Date: Feb. 3, 2011

(54) MODULAR DEVICE FOR POSITIONING AND IMMOBILISATION OF A PATIENT'S BODY FOR SURGICAL OPERATIONS AND CORRESPONDING OPERATING TABLE

(75) Inventors: Uwe Striggow, Aichtal (DE); Michael Paul Witzak, Korb (DE)

Correspondence Address: BIRCH STEWART KOLASCH & BIRCH, PO BOX 747, FALLS CHURCH, VA, 22040-0747, US

(73) Assignee: Schaerer Mayfield Management AG, Munsingen (CH)

(21) Appl. No.: 12/742,396

(22) PCT Filed: Oct. 21, 2008
(86) PCT No.: PCT/CH2008/000438

§ 371 Date: Oct. 20, 2010

(30) Foreign Application Priority Data
Nov. 13, 2007 (EP) PCT/EP2007/062285

(51) Int. Cl. A61F 5/37

(52) U.S. Cl. 128/882

(57) ABSTRACT

This invention relates to a modular device (10) for positioning and immobilisation of a patient's body for surgical operations, which comprises a pelvis support (20) for supporting the patient's pelvis and at least one foot tether support (30) with a foot tether fixation (50) for positioning and immobilisation of a patient's leg, whereby the at least one foot tether support (30) is pivotable in the horizontal and/or in the vertical plane with respect to the pelvis support (20). In addition, the modular device (10) according to the invention can comprise a hip support (60) for positioning the patient's hip during the operation. The invention also relates to an operating table (100) comprising the modular device (10) according to the invention.

### TECHNICAL FIELD

The invention relates to a modular device for positioning and immobilisation of a patient's body for surgical operations and a corresponding operating table equipped with such a modular device. In particular, the invention relates to a modular device for positioning and immobilisation of a patient's body and a corresponding operating table for carrying out minimally invasive surgical interventions on the pelvis and/or limbs of a patient, such as hip joint operations.

### BACKGROUND ART

Used in a known way during operations on the limbs of the skeleton (such as operations on the hip joint) is a traction device for alignment of bones or extension of joints. With this existing technology, only one single movement, which has to be defined before the operation with respect to direction and angle, is possible during the operation. In addition, as no unsterile parts may be touched by the operating surgeon during the operation, on the one hand, and unsterile persons may not come into the vicinity of the operating area on the other hand, it is necessary that control elements for basic setting of the traction device, although well accessible, are positioned in the vicinity of the respective functional component.

Control elements, which are supposed to be manipulated during the operation, must however be positioned sufficiently far away from the operational field. On the other hand, the whole system should require as little space as possible in the operating room in order to ensure good access to the patient and to other installations at any time.

In principle, two systems are currently known and used for solving this problem: telescopic traction devices, on the one hand, in which the control elements are located for the most part in the vicinity of the operating field, and designs with long supports, on the other hand, where the traction device can be shifted and which in many cases require a support floor-side.

In addition, with all systems, it must be possible to achieve a positioning and an immobilisation of the patient's body, desired by the operating surgeon, without limiting the access of the operating surgeon or other installations, in particular image intensifying or image making devices (such as X-ray apparatus or other similar devices), to the required portions of the patient's body. In particular during operations on the pelvis, unobstructed access to the patient's body by X-ray apparatus at all times is of the utmost importance.

EP 1 604 629 A1 (Medacta International S. A.) shows a hospital converter system for surgical intervention on animal body limbs. It comprises an articulated sub-system with an articulation to couple and connect to an extension base with at least two degrees of freedom, a second sub-system of support and movement of the patient limb coupling means with at least two degrees of freedom, a third sub-system associated to the second sub-system, with at least one further degree of freedom, a fourth sub-system of interconnections between the third sub-system and a fifth sub-system. The fourth sub-system provides for one additional degree of freedom, whereas the fifth sub-system provides for three additional degrees of freedom. The disclosed embodiment comprises a carriage interacting with a binary track, whereas the binary track is supported on the floor by means of a pivotable column having a wheel rolling on the ground.

However, this system has a number of disadvantages. First of all, the arrangement of controls is not intuitive which may lead to maloperations by the operating personnel. Furthermore, it will be difficult to carry out refined movements as soon as the pivotable column is completely folded under the binary track and the latter rests on the floor. Further, folding the column requires additional free space in the projection of the operating table. Another disadvantage consists in the limited freedom of the movement of the different parts of the sub-systems which imposes conditions on the possible surgical methods.

New operating techniques on the limbs, especially for hip joint operations, make it necessary for the traction apparatus to be able to be adjusted by personnel inside and outside the operating field in all degrees of freedom, even during the operative intervention. Also for economic reasons, the aim is to be able to use a single device for positioning and immobilisation of the patient's body for all disciplines and for the different schools of orthopaedics and traumatology.

### SUMMARY OF THE INVENTION

Thus the object of this invention is to propose a new and improved device for positioning and immobilisation of a patient's body which does not have the above-mentioned drawbacks of the prior art and which satisfies the requirements of the new, minimally invasive, operating techniques.

According to the present invention, these and other objects are achieved in particular through the features of the independent claims. In addition, further advantageous embodiments follow from the dependent claims and the description.

In particular, the object of the invention is achieved through a modular device for positioning and immobilisation of a patient's body for surgical operations comprising a pelvis support for supporting the patient's pelvis and at least one foot tether support with a foot fixation for immobilisation and positioning of a patient's leg, whereby the at least one foot thether support is pivotable in the horizontal and/or in the vertical plane with respect to the pelvis support. The advantage of such a modular device is, among other things, that the patient's legs (but potentially also arms with a corresponding adapted device) can be easily positioned and immobilised for surgical operations. Moreover, the positioning can be performed not only prior to the operation, but also during the operation in an easy way, also by non-sterile persons. Such a modular device makes it thus possible to perform a broad spectrum of different kinds of operations, including the minimally invasive surgical operations on the hip and/or pelvis of a patient.

In an embodiment variant, the pivoting angle of the at least one foot tether support in the horizontal plane is at least 180°. The particular advantage of this embodiment is, among other things, that the foot tether support can be pivoted basically in any defined position, and also moved completely out of the operating field, if necessary. In particular for the minimally invasive operation techniques, it is necessary to allow a very important horizontal displacement of the patient's legs at any time without complicated manipulations.

In another embodiment variant, the pivoting angle of the at least one foot tether support in the vertical plane is between 20° above the horizontal plane and 55° under the horizontal plane. This embodiment variant has the particular advantage, among other things, that the freedom of movement of the foot tether support can be improved even further. As already indicated in the case of horizontal pivoting, the minimally invasive operation techniques on the limbs and/or pelvis of the patient often require an important displacement of the patient's limbs during the operation. This particular embodiment of the invention thus also allows the carrying out of very demanding and challenging surgical operations.

Preferably, the at least one foot tether support comprises a traction beam that is attached to the pelvis support in such a way that it is pivotable with respect to a horizontal axis as well as a vertical axis, whereas the horizontal axis runs in a plane that is below a support plane for the patient's pelvis. This allows for freely pivoting or turning the patient's leg held by the foot tether support, as required during the surgical operation.

Advantageously, the support beam is pivotably attached with a first end to the pelvis support in a vicinity of the horizontal and vertical pivot axes, whereas a second end of the support beam opposing the first end is supporting the traction beam. Using such a support beam, the need for a supporting element contacting the floor is avoided. Accordingly, the space required in the projection of the operating table is reduced and the traction beam is freely accessible.

In this context, the support beam preferably comprises at least one gas pressure cylinder and a lockable pivot axis for selectively releasing a pivotal movement of the traction beam in a vertical plane. The gas pressure cylinder smoothly controls the pivoting movement of the traction beam about a horizontal axis (i. e. in a vertical plane). In contrast to the known prior art an uncontrolled lowering movement of the support beam is avoided. The lockable pivot axis allows for locking the traction beam in a desired orientation. The lockable axis may be arranged at the first or at the second end of the support beam. It may be constituted e. g. by a spring-loaded excenter locking a bolt of the pivot axis, which may be released by acting against the spring force. The corresponding force may be transmitted e. g. by a bowden cable or a hydraulic system.

In another embodiment variant, the at least one foot tether support comprises a control for controlling the pivoting of the foot tether support. This embodiment variant has the particular advantage, among other things, that the pivoting of the foot tether support can be easily controlled at any time. This control element for controlling the pivoting of the foot tether support can in particular comprise one or more retaining elements which are capable of reliably retaining the foot tether support in the current position, when the foot tether support is to be pivoted. This aspect of this particular embodiment of the present invention is of high importance, as the foot tether support is carrying not only its own weight, but also the weight of a portion of the patient's body during the surgical intervention. Thus, it is of utmost importance to have means which allow an easy pivoting of the foot tether support.

In a further embodiment variant, the at least one foot tether support comprises a telescopic arm and a length adjuster, whereby the length of said at least one foot tether support is adjustable through the actuation of said telescopic arm by means of said length adjuster. The advantage of this embodiment is, inter alia, that the foot tether support can easily be adapted to consistently position and immobilise the leg of any given patient. As different people can have legs of very different lengths, it is very important for the correct immobilisation that the overall length of the foot tether support can be adjusted to fit the given length of the leg. This optimal adjustment to the given length of the patient's leg is a further improvement of the common devices, and enables the carrying out of even the most complex operating techniques in an easy way.

In a further embodiment variant, the foot tether fixation is pivotable and/or rotatable in the horizontal and/or in the vertical plane with respect to the foot tether support. The advantage of this embodiment is, among other things, that the foot of the patient, which has to be immobilised during the surgical operation, can also be moved in different directions during the operation itself Thus, even more complex operating techniques can easily be carried out using this particular embodiment of the present invention.

Preferably, the control element for controlling the pivoting of the foot tether support comprises a handle for controlling a movement of the foot tether support and a lever for releasing a lock for a pivoting movement with respect to a horizontal axis as well as a vertical axis. Advantageously, the handle and the lever are designed and arranged in such a way that they may be simultaneously operated by a single hand of an operator. This allows for an easy and controlled movement of the foot tether support by the operator. The second hand is free to adopt further controlling tasks.

In this context, an operating element for a rotational movement of the foot tether fixation in a vertical plane is designed and arranged in such a way that it may be operated simultaneously with the control element for controlling the pivoting of the foot tether support by the other hand of the operator. Therefore, the operator may usually initiate all the movements required during the surgical operation using his or her hands, without the need for changing controls.

Similarly, an operating element for a linear movement of the foot tether fixation for adduction or abduction of the patient's leg may be designed and arranged in such a way that it may be operated simultaneously with the control element for controlling the pivoting of the foot tether support by the other hand of the operator. If all the three operating elements are designed as described it is possible that the operator may easily carry out all movements required during a surgical procedure by subsequently operating different permutations of two out of the three operating elements.

In another embodiment variant, the foot tether fixation comprises at least one position controller for adjusting the position of the foot tether fixation with respect to the at least one foot tether support. This embodiment variant has the particular advantage, among other things, that a higher flexibility can be attained in positioning and immobilisation of the patient's limbs. As the foot tether fixation can be displaced, the modular device according to this particular embodiment of the invention allows for an optimal hold of the foot and thus for optimal results during surgical operations requiring continuous and repeated displacements of the limbs.

In still another embodiment variant, the device comprises a hip support for positioning of the patient's hip. The advantage of this embodiment is, inter alia, that the patient's hip can be reliably supported and positioned before and during the surgical operations. When the legs must be displaced in different directions, as in the minimally invasive operational techniques on the pelvis, it is important to guarantee a solid support for the hip at all times. In some embodiments, this hip support for positioning of the patient's hips can comprise a fastener for fastening the patient's body and keeping the required position even in the event of very large displacements of the legs. Moreover, the minimally invasive operating techniques on the hip joint may require the displacement of the hips during the operation itself This displacement can easily be achieved by means of this hip support.

In a further embodiment variant, the hip support comprises a hip support cushion and a control element for controlling the position of the hip support cushion with respect to the pelvis support. This embodiment variant has the particular advantage, among other things, that the hip support fulfils two functions at the same time, namely providing a soft support for the patient's body, on the one hand, so as not to cause any grazing, compressions or similar wounds, and, on the other hand, enabling an easy displacement of the hip, during the surgical operation, by personnel located outside the operating area.

In another embodiment variant, the at least one foot tether support is connected to the pelvis support by means of a pivotable connection arm. The particular advantage of this embodiment is, inter alia, that the foot tether support can be pivoted in an even easier way. The combination of the two pivotal movements can make the positioning of the foot tether support even more flexible.

In still another embodiment variant, the at least one foot tether support comprises a lock mechanism for locking the foot tether support in position. This lock mechanism can in particular comprise a lever structure or another similar element which can reliably lock the foot tether support in a given position. This embodiment variant has the particular advantage, among other things, that the foot tether support (and the patient's leg supported by this foot tether support can be reliably locked in a required position, once this position is reached by pivoting of the foot tether support. Since, during the surgical operation, the leg sometimes has to sustain great force, it is very important to be able to guarantee a fixed and solid support of the leg at any time.

In a further embodiment variant, the modular device comprises at least one stationary hip support for stationary support of the patient's hip. This embodiment variant has the particular advantage, among other things, that the patient's hip can also be supported in a stationary way if no displacement of the hip is required during the surgical operation. This can in particular be the case for the pelvis operations or operations on any other portion of the patient's body, or even when carrying out common operation techniques on the limbs of the patient.

In another embodiment variant, the modular device comprises a leg support for stationary support of a patient's leg. The advantage of this embodiment is, among other things, that the modular device according to this embodiment can equally be used for carrying out surgical operations on the limbs of a patient or on another part of the patient's body in which no displacement of the patient's legs are necessary. In this case, the modular device can also be used in a completely stationary way, without any pivoting of its constituent parts.

In yet another embodiment variant, the modular device is at least partially composed of a radiotranslucent material. This embodiment variant has the particular advantage, inter alia, that the modular device according to this particular embodiment of the invention can be used in surgical operations which require making radiographs during the operation. Parts of the patient's body can already be rendered accessible to the X-ray or similar apparatus by the pivoting of the foot tether support and/or displacement of the hip support. However, the use of a radiotranslucent material can facilitate even more the use of such image-making devices during complex surgical operations.

At this point, it should be stated that, besides the modular device for positioning and immobilisation of a patient's body for surgical operations according to the particular embodiments of the invention, the present invention also relates to an operating table comprising a modular device according to the particular embodiments of the present invention.

Other advantageous embodiments and combinations of features come out from the detailed description below and the totality of the claims.

### BRIEF DESCRIPTION OF THE DRAWINGS

The drawings used to explain the embodiments show:

FIG. 1 a perspectival representation of a modular device for positioning and immobilisation of a patient's body for surgical operations according to an embodiment of the present invention;

FIG. 2 a perspectival representation of an operating table comprising a modular device for positioning and immobilisation of a patient's body for surgical operations according to an embodiment of the present invention;

FIG. 3 a perspectival representation of the operating table of FIG. 2 in one possible position during an operation on the hip of a patient;

FIG. 4 a view from above of the operating table of FIG. 3;

FIG. 5 a perspectival representation of the pelvis support in the modular device for positioning and immobilisation of a patient's body for surgical operations according to an embodiment of the present invention;

FIG. 6 a perspectival representation of a foot tether support in the modular device for positioning and immobilisation of a patient's body for surgical operations according to an embodiment of the present invention;

FIG. 7 an enlarged view from above of the end portion of the foot tether support of FIG. 6, comprising a control element for controlling the pivoting of the foot tether support and a length adjuster for adjusting the length of the foot tether support;

FIG. 8 a perspectival representation of the foot tether fixation in the modular device for positioning and immobilisation of a patient's body for surgical operations according to an embodiment of the present invention;

FIG. 9 a perspectival representation of the stationary hip support in the modular device for positioning and immobilisation of a patient's body for surgical operations according to an embodiment of the present invention;

FIG. 10 a perspectival representation of the leg support in the modular device for positioning and immobilisation of a patient's body for surgical operations according to an embodiment of the present invention;

FIG. 11 a perspectival representation of the hip support in the modular device for positioning and immobilisation of a patient's body for surgical operations according to an embodiment of the present invention;

FIG. 12 a perspective view of a foot tether support according to a second embodiment of the present invention;

FIG. 13 a perspective view of the foot-tether fixation of the foot tether support according to the second embodiment; and

FIG. 14 a perspective view of the linear compensation element of the foot-tether fixation according to the second embodiment, whereas some elements of the compensation element are exposed.

In the figures, the same components are given the same reference symbols.

### PREFERRED EMBODIMENTS

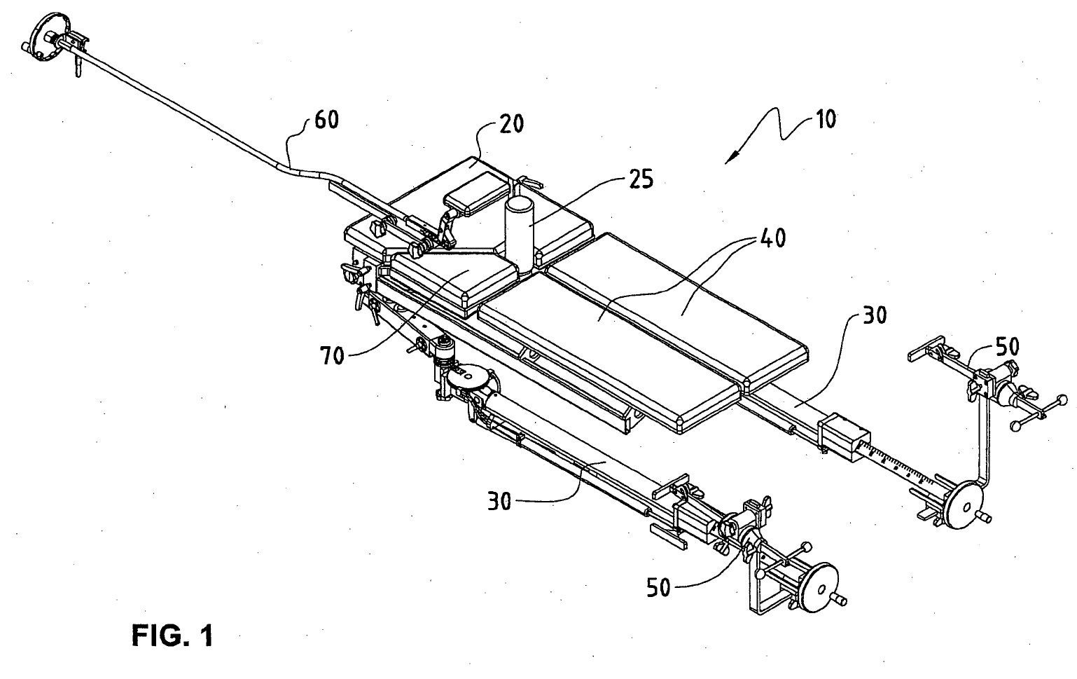

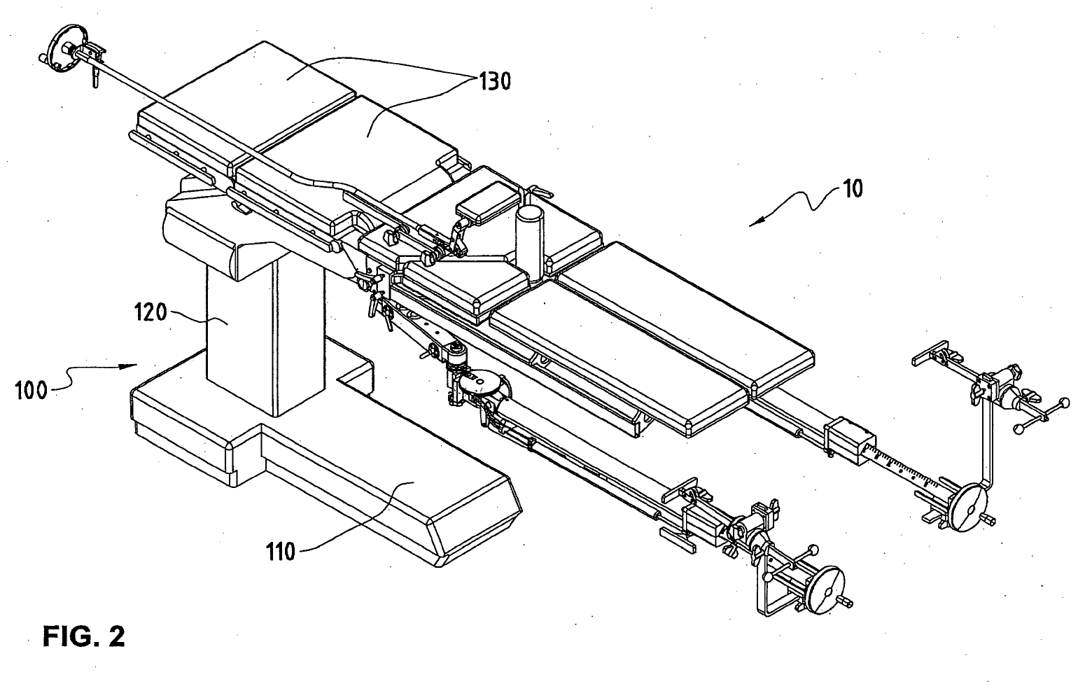

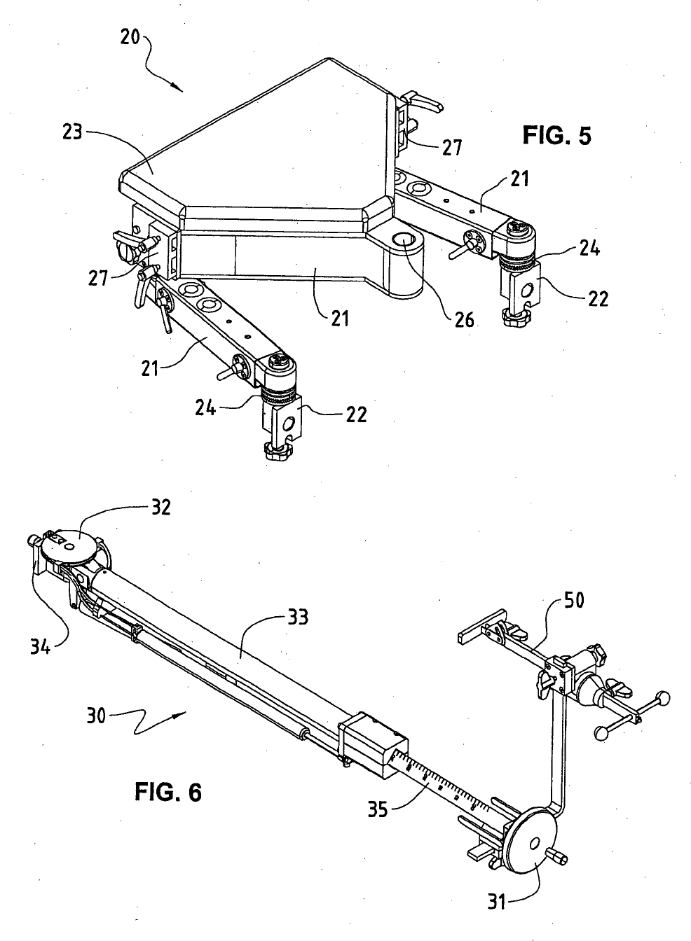

FIG. 1 illustrates a modular device 10 for positioning and immobilisation of a patient's body for surgical operations according to an embodiment of the present invention. In FIG. 1, the reference numeral 20 refers to a pelvis support, which can be used for positioning of the patient's pelvis before, during and after the surgical operation. The modular device 10 is designed in particular such that it can be mounted on operating tables provided therefore (as will be illustrated in FIG. 2) or also on conventional operating tables. For this purpose, the operating table and the pelvis support 20 can be provided with corresponding connectors (not shown). The pelvis support 20 can in particular also comprise a counter-traction holder 25 which can hold the patient's body in a given position when one or both legs, or the hip of the patient are displaced during the surgical operation. The structure of a pelvis support 20 of the modular device 10 according to a particular embodiment of the present invention will be described in more detail below, with respect to FIG. 5.

The reference numeral 30 in FIG. 1 refers to a foot tether support which can position and immobilise the legs of the patient before and during the surgical operation. The foot tether support 30 can in particular be designed and installed in such a way that they are telescopic systems which can be managed without any additional support and on which all control elements are installed outside the operating field. The structure of a pelvis support 30 of the modular device 10 according to a particular embodiment of the present invention will be described in more detail below, with respect to FIG. 6.

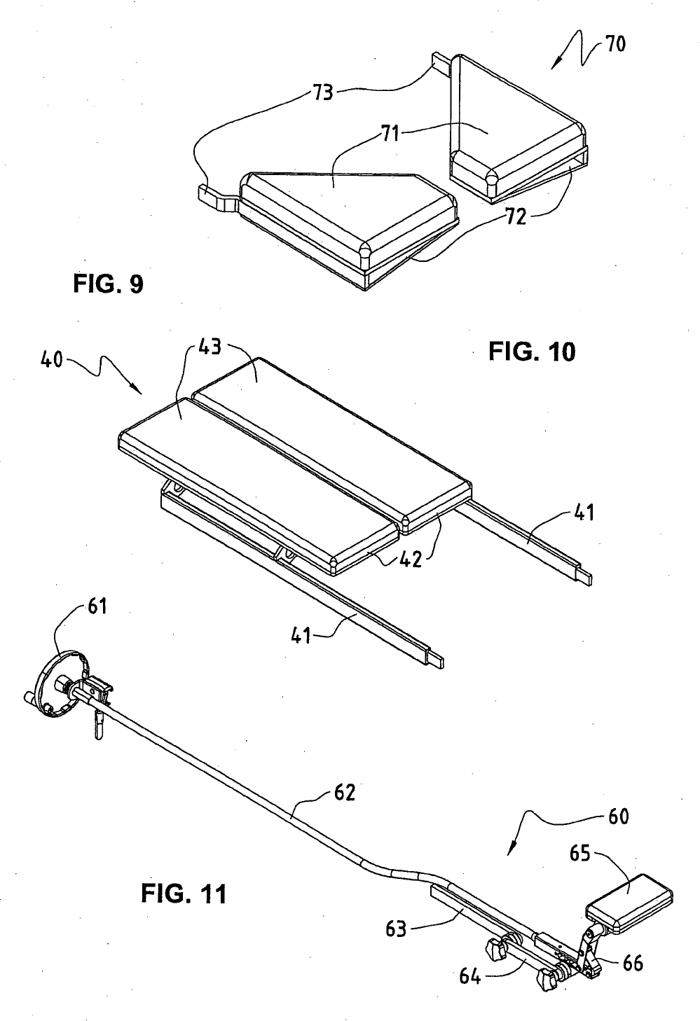

The hip support, stationary hip support and leg support are represented in FIG. 1 by the reference numerals 60, 70 and 40, respectively. The hip support 60 supports the hip of the patient when it has to be displaced (in particular with respect to height) during the operation. The stationary hip support 70, on the other hand, support the hips of the patient in a stationary way, and this way make possible the creation of a closed lying surface for the patient, in collaboration with the pelvis support 20. The leg support 40 serves the stationary positioning of the legs of the patient before or during the surgical operation, when the operating surgeon thinks it necessary. The structure of the hip support 60, stationary hip support 70 and leg support 40 of the modular device 10 according to a particular embodiment of the present invention will be described in more detail below, with respect to FIGS. 9, 10 and 11.

Finally, the reference numeral 50 in FIG. 1 refers to two foot tether fixations. It is perfectly clear for any person skilled in the art, however, that the modular device 10 according to the invention can also have only one single foot tether fixation, if necessary. The foot tether fixations are each mounted on a foot tether support 30, and thus allow, in connection with the respective foot tether support 30, a correct positioning and immobilisation of the patient's foot during the operation. FIG. 8 illustrates in detail the structure of the foot tether fixation 50.

FIG. 2 shows in a schematic way an operating table 100 which is equipped with a modular device 10 according to an embodiment of the invention shown in Figure I. For this purpose, the operating table 100 is provided with connectors (not shown) for attaching and mounting the modular device 10 to the operating table 100 in a removable way. These connectors are designed and placed in such a way as to allow an easy and stable connection of the modular device 10 to the table 100. Any common operating table can, in principle, be equipped with the corresponding connectors in order to be used in combination with the modular device 10 according to the invention.

The reference number 110 in FIG. 2 refers to a base plate by means of which the operating table TOO is placed on the floor, a central support 120 for connecting the base plate with a lying down area 130, on which the patient can lie down during the surgical operation. The lying down area 130 can in particular be supplemented by the pelvis support, stationary hip support and/or leg support of the modular device 10. Moreover, the lying down area 130 can be composed of various movable segments, such that an optimal positioning of the patient's torso can be guaranteed at any time. The central support 120 can in particular have a height adjusting module (not shown) for adjusting the height of the lying down area 130 with respect to the base plate 110.

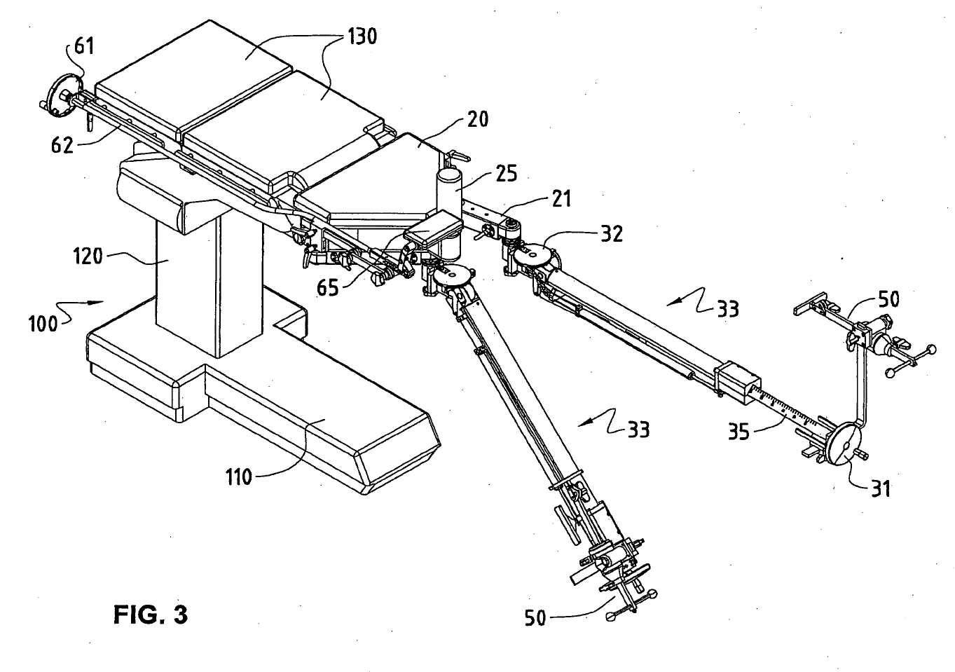

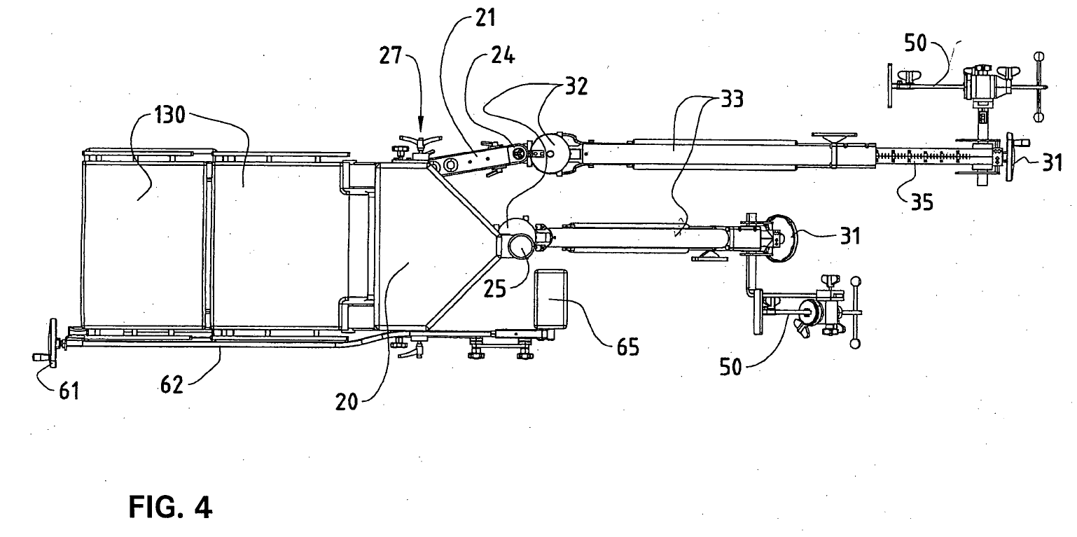

FIG. 3 shows the operating table 110 in one possible position during an operation on the hip of a patient. The elements which were described with respect to FIGS. 1 and 2 are referred to with the same reference numerals. The identical configuration of the operating table 110 in a view from above is represented in FIG. 4. The following description relates therefore to both Figures.

The modular device 10 has been mounted on the operating table 100 in FIG. 3. The stationary hip support and the leg support of the modular device 10 have been removed, as they are not required for this type of operation. It is however clear to any person skilled in the art that the fourth and fifth supporting devices can be used again, if required by the operation. The hip support 60 for dynamic positioning of the hip of the patient has been mounted on the pelvis support 20 on one side of the pelvis support 20, instead of the stationary hip support 70 for static positioning of the patient's hip by means of the hold point 27. By means of the control element 61 and the force transmission 62, the hip support cushion 65 has been moved into a position required by the operation. Moreover, the connection arm 21 on the same side of the pelvis support 20 has been pivoted 1 towards the vertical midplane of the modular device 10 in order to position the corresponding foot tether support 30 in the required position. Moreover, this foot tether support 30 has been pivoted around the pivoting joint 32 with respect to the vertical plane in the direction towards the floor. Finally, the other foot tether support 30 has been extended by means of the length adjuster 31, as the telescopic arm 35 of this foot tether support 30 has been pulled out. This configuration of the modular device 10 allows for an easy operation of the hip joint according to a minimally invasive operation technique.

FIG. 5 shows the pelvis support 20 of the modular device 10 for positioning and immobilisation of a patient's body for surgical operations according to an embodiment of the invention. The pelvis support 20 takes basically the form of a base plate 25 with a cushion 23 positioned on top of it. The cushion 23 can in particular be disposed on the base plate 25 in a removable way. Installed below and mounted to the base plate 25 are two connection arms 21, which can be pivoted around their fixing point by a 360° angle. Each connection arm 21 is connected to a connector 22 for connecting the foot tether support to the connecting arm 21 and, therefore, to the pelvis support 20.

The connection between the connecting arm 21 and the connector 22 is achieved by means of an articulation or joint 24 which allows pivoting by an angle of 360° around the middle axis. Each articulation 24 can be locked in a play-free way with a toothed wheel locking device or another similar device. This locking unit of the articulation 24 is designed such that the respective weights of the connection arms 21, foot tether support 30 and of the patient's legs do not have to be lifted at any time. To this end, a particular locking and holding mechanism is provided (not shown). The pivotable connection arms 21, among other things, make it possible for the user to position all nonradiotranslucent parts of the device 10 outside the later necessary X-ray passage before or even during the surgical operation. Placed on the sides of the base plate 25 are the hold points 27 for the stationary hip support and leg support (not shown). Thus it is possible, with the cushion 23 placed on, to pivot the connecting arms 21 with the connected second supporting devices 30 freely. Moreover, integrated in the base plate 21 is a receiving element 26 for the counter-traction holder 25 for holding the patient's body in position when displacing the legs.

The foot tether support 30 according to one embodiment of the present invention is illustrated in detail in FIG. 6. The foot tether support 30 consists essentially of a traction beam 33 with a connecting joint 32 which allows for the pivoting of the foot tether support 30 in horizontal and/or vertical plane. The connecting joint 32 is arranged below the support plane for the patient's pelvis defined by the pelvis support 20, i. e. axes for pivoting the traction beam 33 in the horizontal plane as well as in the vertical plane basically intersect in a point that is below the support plane. The geometry is chosen in such a way that the patient's leg held by the foot tether support 30 may be freely pivoted and/or turned, as required during the surgical operation. The traction beam 33 contains a telescopic arm 35 which can be controlled by means of the length adjuster 31. The foot tether fixation 50 is mounted on the end portion of the foot tether support 30.

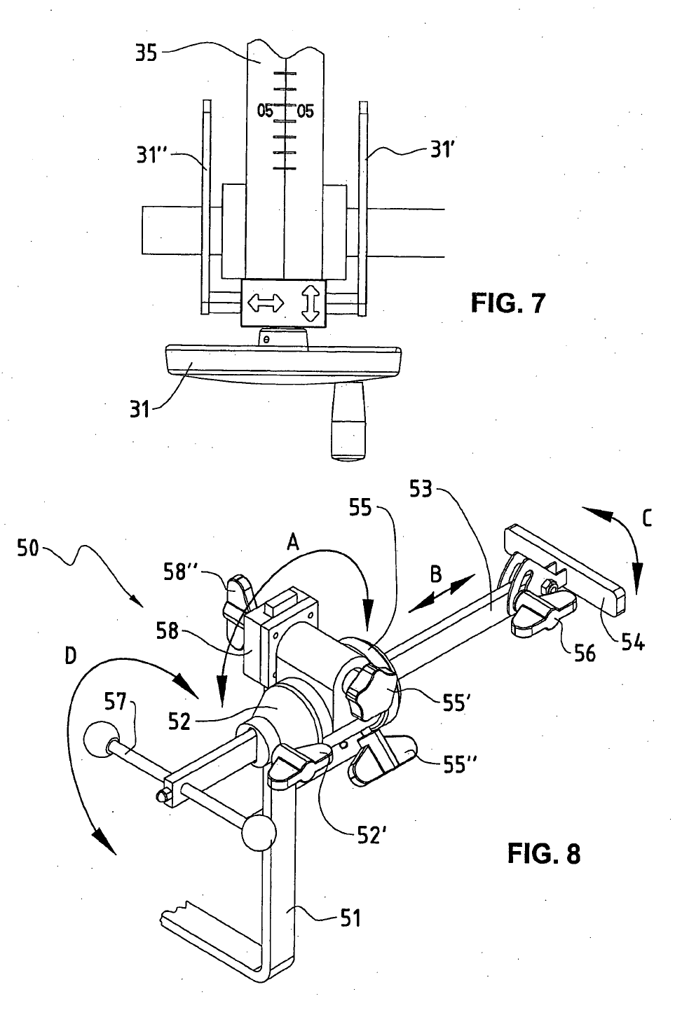

The foot tether support 30 is able to be adjusted in length by means of the length adjuster 31, and thus allows an easy adaptation to the body height of the patient. In addition, both traction force and compressive force can be exerted in this way on parts of the patient (in particular on the legs) during certain operations. If, for reasons of change of traction direction or of repositioning of the patient, the vertical angle of the traction beam 33 has to be changed, this can be done in a continuously adjustable way from 20° over the plane of the lying surface of the operating table 100 to 55° under this plane by releasing the vertical immobilization of the foot tether support 30 by means of actuation of the corresponding lock mechanism 31″, which is shown in detail in FIG. 7. Horizontally, the foot tether support 30 is able to pivot by 90° to the left and right in each case, when the corresponding lock mechanism 31′ is actuated. In addition, the lock mechanism 31′ can be brought into a position which allows the horizontal immobilization to be kept released, so that the second supporting device is freely movable in this plane.

A foot tether fixation 50 according to a particular embodiment of the invention is illustrated in FIG. 8. The connecting element 51 between the foot tether support 30 and the foot tether fixation 50 serves the purpose of placement, and makes possible a variable position of the foot tether fixation 50 relative to the foot tether support 30, so that the parts of the patient to be operated on are optimally accessible to the operating surgeon and other medical devices. The connecting element 51 is dimensioned such that it can be inserted in the foot tether support 30 with each of the legs of different length, from the left or from the right, the free leg in each case pointing upward or downward.

With the sliding piece 58 and the first clamping screw 58″, the foot tether fixation 50 is attached on the connecting element 51 at the desired height relative to the foot tether support 30. The basic unit 52 of the foot tether fixation 50 allows itself to be pivoted by 360° relative to the connecting element 51 in direction A (represented in FIG. 8 by an arrow). The linear compensation element 53 is easily displaceable toward the basic unit 52 in direction B (also represented by an arrow) by means of a roller guide in order to prevent undesired traction forces on the patient. If tractionforces are desired, however, the linear compensation element 53 can be fixed in any position via a second clamping screw 52′. The connecting element for the patient tether 54 is pivotable in direction C (as represented by the corresponding arrow) and direction A when the third clamping screw 56 is released, to enable an optimal alignment of the linear compensation element 53 with respect to the patient. The linear compensation element 53 is in addition rotatable in direction D (again represented by an arrow) by 360°, using a handle 57 in the case of increased force requirement. An adjustable free movement 55 (with the positions left, free and right) is integrated into the foot tether fixation 50 to facilitate the manipulations. The rotation can be locked via a fourth clamping screw 55″.

FIGS. 9 and 10 show the stationary hip support 70 and leg support 40 of the modular device 10 according to an embodiment of the present invention. The stationary hip support 70 has connectors 73 on both sides for being connected to the hold points 27 of the pelvis support 20. They make possible the creation of a closed lying sutface for the patient. The supports 72 of the stationary hip support 70 can in particular consist of a radiotranslucent material in the area of the X-ray passage. The upper side of the stationary hip support 70 is preferably covered with two removable cushion supports 71.

The leg support (the leg support cushions) 40 are likewise fixed in the hold points 27 of the pelvis support 20 by means of the connection holders 41. The leg support 40 serve the stationary positioning of the legs of the patient before the operation or during the operation, if the operating surgeon thinks it necessary. The upper side of the leg support 40 is covered in principle with two removable cushion supports 43. In a particular embodiment of the invention, the supports 42 of the leg support 40 can also consist of a radiotranslucent material in the area of the X-ray passage.

The hip support 60 is illustrated in FIG. 11. This hip support (or femoral support) is meant to support, position and immobilise the hip of the patient during the surgical operation. It can be fixed to thebase plate 25 of the pelvis support 20 (left, right, or on both sides), instead of the stationary hip support 70. This hip support is then located directly below the covered operating field, and holds the thigh and the hip of the patient at the height desired by the operating surgeon during the operational intervention. The hip support 60 comprises at least one control element 61 for controlling the height of the hip supporting cushion 65. Since this control element 61 is located outside the operating field, the adjustment of the height can be performed by any auxiliary person also during the operation since entry into the operating field is not necessary.

The structural parts under the hip support cushion 65 can in particular be made of a radiotranslucent material since, as a rule, directly after placement of a hip prosthesis in a hip joint operation, the position of the prosthesis is checked with an imaging technique (such as X-ray or similar). The height of the hip support cushion 65 can be adapted with the height adjustment unit 66 by means of the lever for the force transmission 62 after the actuation by means of the control element 61. The hip support 60 according to the particular embodiment of the invention can also comprise a positioning unit 63 which makes it possible, in combination with the connecting element 64 for connecting the hip support to the pelvis support 20, to bring the hip support 60 into a position which is well adapted to the anatomy of the patient before the operation.

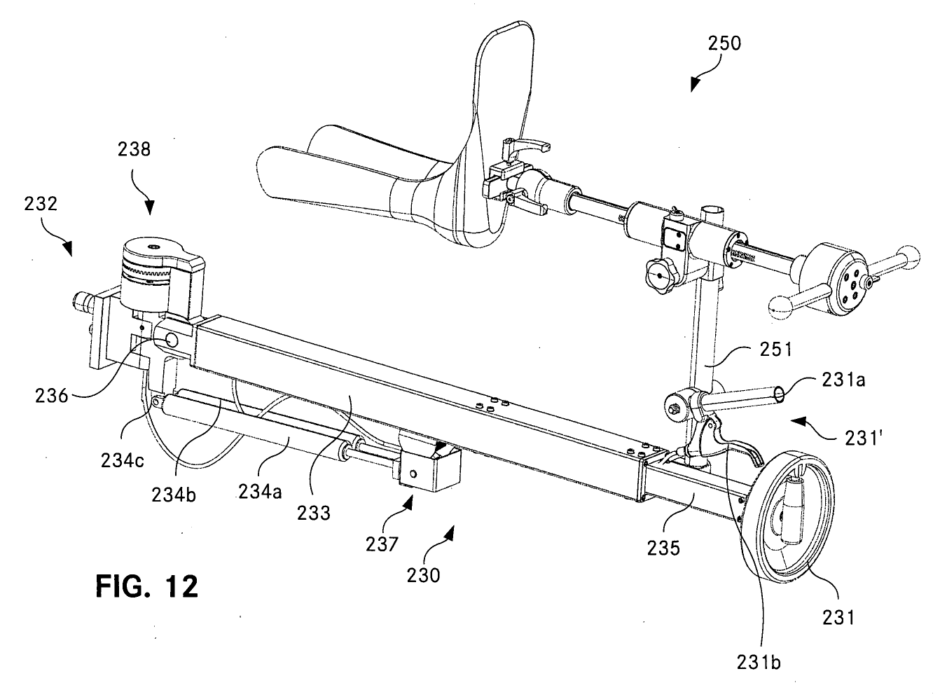

FIG. 12 shows a perspective view of a foot tether support according to a second embodiment of the present invention. The foot tether support 230 may be used in connection with a first, third, fourth and/or fifth supporting element as described above, in connection with FIGS. 1-5 and 9-11, i. e. the foot tether support 230 may be attached to the connector 22 of the connection arms 21. With respect to the first embodiment, the controls of this second embodiment are optimized in view of ergonomics as well as the prevention of handling errors.

Again, the foot tether support 230 serve for positioning and immobilizing the legs of the patient before and during the surgical operation. Again, the foot tether support 230 comprises a telescopic system which can be managed without any additional support. For that purpose, it comprises two gas pressure cylinders 234a, 234b which are pivotably connected to a pivot axis 234c arranged on the connecting joint 232, below the horizontal pivot axis 236 for the traction beam 233. At their distal ends, the gas pressure cylinders 234a, 234b are connected to a lockable pivot axis 237, which comprises an excenter, which is fixedly connected to the gas pressure cylinders 234a, 234b, interacting with a central bolt being fixedly connected to an attachment frame. The attachment frame is affixed to the housing of the traction beam 233. Normally, the excenter is pressed against the bolt by means of a spring. However, by means of a bowden cable, the excenter may be rotated against the force of the spring, thereby unlocking the bolt. This allows for pivoting the gas pressure cylinders 234a, 234b with respect to the traction beam 233 and therefore pivoting the traction beam 233 with respect to the connecting joint 232, about a horizontal axis, i. e. in a vertical plane.

The connecting joint 232 further allows for pivoting the traction beam 33 about a vertical axis, i. e. in a horizontal plane. Again, this pivoting movement may be locked. For that purpose, the connecting joint 232 comprises a brake device 238, featuring two toothed disks being pressed against each other by means of springs. By means of a bowden cable, the toothed disks may be separated from each other such that a relative rotational movement of the disks and therefore rotation of the traction beam 233 about the vertical axis is enabled.

Again, the traction beam 233 features a telescopic arm 235 including a spindle that may be actuated by a rotating disk 231 attached to the free end of the spindle. Thereby, the foot tether support 230 is able to be adjusted in length and thus allows an easy adaptation to the body height of the patient. In addition, both traction force and compressive force can be exerted in this way on parts of the patient (in particular on the legs) during certain operations. If, for reasons of change of traction direction or of repositioning of the patient, the vertical angle of the traction beam 233 has to be changed, this can be done in a continuously adjustable way from 20° over the plane of the lying surface of the operating table 100 to 55° under this plane. Horizontally, starting from a middle position, the foot tether support 30 is able to pivot by 90° to the left and right in each case.

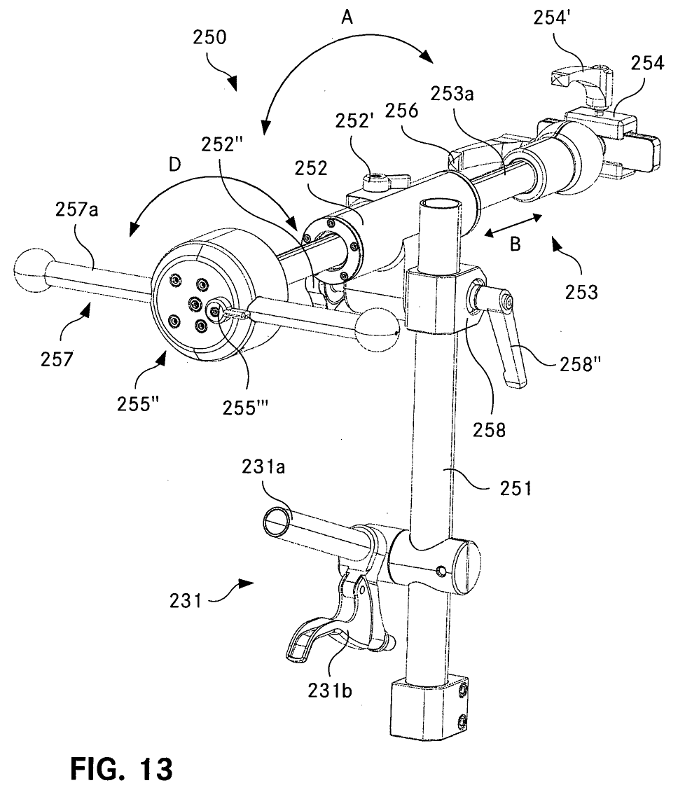

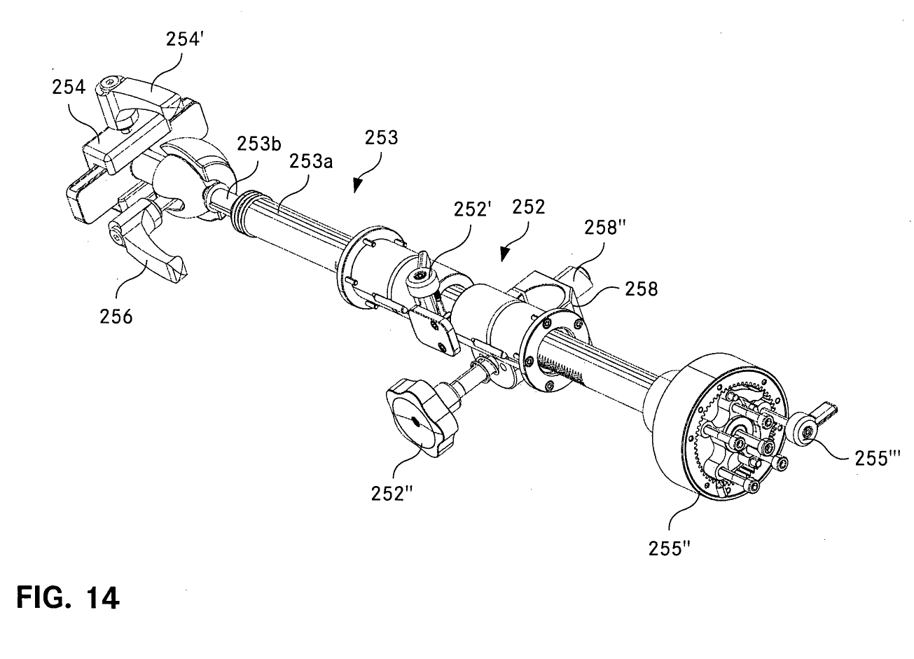

Attached to the telescopic arm 235 of the traction beam 233 is a foot tether fixation 250. The foot tether fixation 250 allows, in connection with the respective foot tether support 230, a correct positioning and immobilisation of the patient's foot during the operation. FIG. 13 shows a perspective view of the foot-tether fixation 250 of the foot tether support according to the second embodiment. FIG. 14 shows a perspective view of the linear compensation element of the foot-tether fixation 250 according to the second embodiment, whereas some elements of the compensation element are exposed.

The foot tether fixation 250 comprises a vertical connecting element 251 constituted by a pipe profile. A lock mechanism 231′ comprising a grip 231a as well as a lever 231b which may be pressed against the grip 231a is attached to the vertical connecting element 251. A bowden cable is attached to the grip 231a. It is guided within the extension beam 233 to the lockable pivot axis 237 as well as to the brake device 238. Therefore, by pressing the lever 231b against the grip 231a, which may be easily done with a single hand, both the lock of the pivot axis as well as of the vertical axis are released and the extension beam 233 may be pivoted about the vertical axis as well as about the horizontal axis. The latter movement is controlled by the gas pressure cylinders 234a, 234b.

A sliding piece 258 is attached to the vertical connecting element 251 of the foot tether fixation 250. It may be moved along the vertical connecting element 251 and locked by means of a clamping screw 258″. A basic unit 252 of the foot tether fixation 250 allows itself to be pivoted by 360° relative to the sliding piece 258 in direction A (represented in FIG. 13 by an arrow). The rotational orientation of the basic unit 252 may be locked by means of a clamping screw 252″. A linear compensation element 253 is linearly guided within the basic unit 252, allowing for displacement along direction B. For guiding the linear compensation element 253 a number of roller cages are accommodated within the housing of the basic unit 252, guiding a hollow profile 253a having a circular cross-section. On its outer surface, the hollow profile 253a features a gearing that interacts with a releasable ratchet. The ratchet is operated by a lever 252′ arranged on top of the housing of the basic unit 252. The ratchet may be set to different operational positions, i. e. locked both ways, released, locked in one direction/released in the other direction. Releasing the linear compensation element 253 allows for preventing undesired traction forces on the patient. If traction forces are desired, however, the linear compensation element 253 can be fixed in any position via the ratchet.

Inside the hollow profile 253a a spindle 253b is accommodated. On a first end, the spindle is connected via a further ratchet mechanism 255″ with a handle 257, comprising two gripping levers 257a, each having a length of 150 mm. The further ratchet mechanism 255″ is a rotational ratchet known as such, comprising ratchet elements interacting with a crown gear enclosing the ratchet elements. On a second end, the spindle is connected to a ball interacting with a socket. The ball and socket joint allows for freely pivoting the connecting element 254 for the patient tether in order to enable an optimal alignment of the linear compensation element 253 with respect to the patient. The pivoting may be inhibited by tigthening a further locking screw 256, tightening the socket, which is constitued of two relatively movable parts, against the ball. Connected with the ball part of the ball and socket joint is a slidable connecting element 254. Its linear position may be locked by a further locking screw 254′. As soon as the ratchet mechanism 255″ is released by using a lever 255′″ (again, the mechanism features a number of different operational positions) the connecting element 254 may be rotated by 360° about axis D. The length of the gripping levers 257a allows for a reduction of the required rotation force.

In the course of a surgical operation, the position of the foot tether fixation 250 will usually be adapted to the physiology of the patient as well as the requirements of the operation procedure by adjusting the height of the sliding piece 258 relative to the vertical connecting element 251 (locked by the clamping screw 258″), the rotational position of the basic unit 252 relative to the sliding piece 258 (locked by the clamping screw 252″), as well as the orientation and lateral position of the connecting element 254 (locked by clamping screws 256, 254′). Usually, these adjustments will be maintained during the operation procedure. However, using the lock mechanism 231′, the handle 257 as well as the levers 255′″, 252′ the foot tether may still be flexibly moved in an easily controllable way. These ergonomic controls allow for rotating the foot tether around three pivot axes that are perpendicular to each other as well as adjusting the extension. Due to the simple structure and layout of the controls as well as their size, maloperations, especially in a stressful environment, are avoided.

The components of the foot tether support according to the second embodiment are designed in such a way that they may carry forces in the direction of the main extension of the supported limb of at least 500 N as well as at least 600 N in a direction perpendicular to this main extension. These forces have to be borne in the context of novel surgical operation, e. g. with minimally invasive arthroscopy.

Although the present disclosure has been described with reference to particular means, materials and embodiments, one skilled in the art can easily ascertain from the foregoing description the essential characteristics of the present disclosure, while various changes and modifications may be made to adapt the various uses and characteristics without departing from the spirit and scope of the present invention as set forth in the following claims.

1.-21. (canceled)

22. A modular device for positioning and immobilisation of a patient's body for surgical operations, characterised in that it comprises a pelvis support for supporting the patient's pelvis and at least one foot tether support with a foot tether fixation for immobilisation and positioning of a patient's leg, whereby the at least one foot tether support is pivotable in the horizontal and/or in the vertical plane with respect to the pelvis support.

23. The modular device according to claim 22, characterised in that the pivoting angle of the atleast one foot tether support in the horizontal plane is at least 180°.

24. The modular device according to claim 22, characterised in that the pivoting angle of the at least one foot tether support in the vertical plane is between 20° above the horizontal plane and 55° under the horizontal plane.

25. The modular device according to claim 22, characterized in that the at least one foot tether support comprises a traction beam that is attached to the pelvis support in such a way that it is pivotable with respect to a horizontal axis as well as a vertical axis, whereas the horizontal axis runs in a plane that is below a support plane for the patient's pelvis.

26. The modular device according to claim 25, characterized by a support beam being pivotably attached with a first end to the pelvis support in a vicinity of the horizontal and vertical pivot axes, whereas a second end of the support beam opposing the first end is supporting the traction beam.

27. The modular device according to claim 26, characterised in that the support beam comprises at least one gas pressure cylinder and a lockable pivot axis for selectively releasing a pivotal movement of the traction beam in a vertical plane.

28. The modular device according to claim 22, characterised in that the at least one foot tether support comprises a control element for controlling the pivoting of the foot tether support.

29. The modular device according to claim 22, characterised in that the at least one foot tether support comprises a telescopic arm and a length adjuster, whereby the length of said at least one foot tether support is adjustable through the actuation of said telescopic arm by means of said length adjuster.

30. The modular device according to claim 22, characterised in that the foot tether fixation is pivotable and/or rotatable in the horizontal and/or in the vertical plane with respect to the foot tether support.

31. The modular device according to claim 28, characterised in that the control element for controlling the pivoting of the foot tether support comprises a handle for controlling a movement of the foot tether support and a lever for releasing a lock for a pivoting movement with respect to a horizontal axis as well as a vertical axis, whereas the handle and the lever are designed and arranged in such a way that they may be simultaneously operated by a single hand of an operator.

32. The modular device according to claim 31, characterised in that an operating element for a rotational movement of the foot tether fixation in a vertical plane is designed and arranged in such a way that it may be operated simultaneously with the control element for controlling the pivoting of the foot tether support by the other hand of the operator.

33. The modular device according to claim 31, characterised in that an operating element for a linear movement of the foot tether fixation for adduction or abduction of the patient's leg is designed and arranged in such a way that it may be operated simultaneously with the control element for controlling the pivoting of the foot tether support by the other hand of the operator.

34. The modular device according to claim 22, characterised in that the foot tether fixation comprises at least one position controller for adjusting the position of the foot tether fixation with respect to the at least one foot tether support.

35. The modular device according to claim 22, characterised in that the at least one foot tether support is connected to the pelvis support by means of a pivotable connection arm.

36. The modular device according to claim 22, characterised in that the at least one foot tether support comprises a lock mechanism for locking the foot tether support in position.

37. The modular device according to claim 22, characterised in that the device comprises a hip support for positioning of the patient's hip.

38. The modular device according to claim 37, characterised in that the hip support comprises a hip support cushion and a control element for controlling the position of the hip support cushion with respect to the pelvis support.

39. The modular device according to claim 22, characterised in that it comprises at least one stationary hip support for stationary support of the patient's hip.

40. The modular device according to claim 22, characterised in that it comprises a leg support for stationary support of a patient's leg.

41. The modular device according to claim 22, characterised in that it is at least partially composed of a radiotranslucent material.

42. An operating table comprising a base plate, a central support, and a lying down area, characterised in that it further comprises a modular device according to claim 22.
